(19) United States
(12) Patent Application Publication
(10) Pub. No.: US 2026/0083454 A1
(43) Pub. Date: Mar. 26, 2026

(54) ADJUSTABLE MEDICAL/VETERINARY SUTURE CRIMP TOOL

(71) Applicant: MWI Veterinary Supply Co., Boise, ID (US)

(72) Inventors: Graham Smith, Newburyport, MA (US); Christian Wagner, Königsfeld/Neuhausen (DE); Thomas Butsch, Rietheim/Weilheim (DE); Stephen Michael Fitzpatrick, Upton, MA (US)

(21) Appl. No.: 19/410,187

(22) Filed: Dec. 5, 2025

Related U.S. Application Data
(63) Continuation-in-part of application No. 18/657,212, filed on May 7, 2024.
Application No. 18/657,212 is a continuation-in-part of application No. 18/456,634, filed on Aug. 28, 2023.
(60) Provisional application No. 63/477,671, filed on Dec. 29, 2022.

(51) Int. Cl. A61B 17/04; A61B 90/00

(52) U.S. Cl. CPC A61B 17/0487; A61B 2017/0488; A61B 2090/034; A61B 2090/0811

(57) ABSTRACT

An adjustable medical/veterinary suture crimp tool system includes a crimp tool with a stationary jaw at a distal end of fixed shank, a moveable jaw at a distal end of a linearly driven crossbar for deforming a crimp tube located between the stationary jaw and the moveable jaw, and an alignment fiducial associated with at least one jaw. A first handle is connected to a fixed shank, and a second handle is pivotably connected to the fixed shank and configured to drive the crossbar linearly with respect to the fixed shank to crimp the crimp tube. A stop is configured to limit the extent of travel of the crossbar and a mechanism cooperates with the stop for adjusting the extent of travel of the crossbar for crimp tube deformation. An internally threaded crimp tube has an outer alignment fiducial for alignment with the alignment fiducial of the at least one said jaw of the crimp tube.

### RELATED APPLICATIONS

This application is a continuation in part of and claims benefit of and priority to U.S. patent application Ser. No. 18/657,212 filed May 7, 2024, which itself is a continuation in part of and claims benefit of and priority to U.S. patent application Ser. No. 18/456,634 filed Aug. 28, 2023, which itself claims the benefit of and prior to U.S. Provisional Application Ser. No. 63/477,671 filed Dec. 29, 2022, under 35 U.S.C. §§ 119, 120, 363, 365, and 37 C.F.R. § 1.55 and § 1.78, and all of which are incorporated herein by this reference.

### FIELD OF THE INVENTION

This subject disclosure relates to crimping devices typically used in connection with crimp tubes used in medical and veterinary procedures.

### BACKGROUND OF THE INVENTION

Crimping devices and crimp tubes are used in procedures such as cruciate ligament stabilization where ends of the suture (e.g., a mono-filament) are held together in a crimp tube or clamp. A crimping device deforms a crimp tube and locks the suture in therein. See U.S. Pat. Nos. 6,905,010; 8,495,902 and U.S. Patent Publication No. 2009/0248063 all incorporated herein by this reference.

It is important that the crimping action be precise. Too much press on the suture can cause breakages of the suture. If the crimp tube is not deformed enough, on the other hand, the suture can slip in the crimp tube. See also U.S. Pat. Nos. 7,121,307 and 6,152,188 and Published U.S. Application No. 2006/0156784, all incorporated herein by this reference.

Different suture sizes are typically used for different size patients (e.g., dogs). Suture ranges in size from 20 lb to 100 lb. A 100 lb suture is stronger and often larger in diameter than a 20 lb suture.

Crimp tube dimensions vary as well. Usually, a crimp tube for 100 lb suture has a thicker wall thickness than a crimp tube for a 20 lb suture.

The crimp tool may have a fixed stop which, at the full stroke of the device, the jaws on each side of the crimp tube are always the same distance apart.

For thicker walled crimp tubes, it can be difficult for some medical personnel to even reach the crimper stop.

### SUMMARY OF THE INVENTION

Featured is a new adjustable suture crimp tool. Further featured is such an adjustable suture crimp tool which makes it easier for medical personnel to fully and properly crimp the crimp tube about sutures of different sizes.

Also featured is a crimp tube with a constant, fairly small wall thickness used for all suture sizes thereby making it easier for medical personnel to fully and properly crimp the crimp tube. Preferably, the crimp tube has internal threads for better gripping of the suture(s) and an external alignment fiducial alignable with a corresponding alignment fiducial of the crimp tool.

Herein, a new system is proposed where the different sizes crimp tube all have the same small wall thickness which enables easier crimping. And to better ensure a proper crimp about the suture, the crimp tool has a variable stop set by the practitioner to match the chosen suture (and crimp tube) size. Thus, in one example, for a 20 lb suture, the crimp tube is deformed to the same degree as a crimp tube for a 100 lb suture.

Featured is an adjustable medical/veterinary suture crimp tool comprising a stationary jaw at a distal end of fixed shank, a moveable jaw at a distal end of a linearly driven crossbar for deforming a crimp tube located between the stationary jaw and the moveable jaw, a first handle connected to a fixed shank, and a second handle pivotably connected to the fixed shank and configured to drive the crossbar linearly with respect to the fixed shank to crimp the crimp tube. A stop is configured to limit the extent of travel of the crossbar and a mechanism, cooperating with the stop, adjusts the extent of travel of the crossbar for crimp tube deformation. An alignment fiducial is associated with at least one jaw of the crimp tool. A crimp tube also includes an alignment fiducial and internal suture gripping surfaces.

In one embodiment, the stop is on the fixed shank, and the mechanism includes a dial on the crossbar with edge recesses of different depths which engage the stop. The dial may include a central post extending through the linearly driven crossbar. The fixed shank may include at least one spring loaded bearing and the dial includes one or more indentations receiving the spring loaded bearing therein for providing tactile feedback.

In one example, at least one jaw includes an upwardly extending nose extension.

In other embodiments, the stop is on the first handle and extends inwardly towards the second handle and the mechanism is a button or wheel which changes the length of the stop relative to the second handle.

In one example, the stationary jaw at the distal end of the fixed shank has across its width and extending at least partially along its length spaced fixed crimp members including at least first, second, and third crimp members and the movable jaw at the distal end of the linearly driven crossbar has across its width and extending at least partially along its length, spaced crimp members including at least fourth, fifth, and sixth crimp members opposing, respectively the first, second, and third crimp members of the stationary jaw. The crimp tool alignment fiducial may be associated with said fifth crimp member of the stationary jaw for alignment with the alignment fiducial of the crimp tube.

Also featured is an adjustable medical/veterinary suture crimp tool comprising a first arm having a proximal first handle and a distal first jaw for crimping an internally threaded crimp tube with an outer alignment fiducial. A second arm is pivotably coupled to the first arm and has a proximal second handle and a distal second jaw for crimping the crimp tube. At least one jaw also has an alignment fiducial. A stop is configured to limit the extent of travel of the first arm relative to the second arm, and a mechanism, cooperating with the stop, configured to adjust the extent of travel of the first arm relative to the second arm for crimp tube deformation.

In one example, the first arm includes a fixed shank terminating in the first jaw and the second arm includes a linearly driven crossbar terminating in the second jaw.

Also featured is an adjustable/medical veterinary suture crimp tool system comprising a crimp tool with the first arm having a proximal first handle and a distal first jaw for crimping an internally scored crimp tube, a second arm pivotably coupled to the first arm and having a proximal second handle and a distal second jaw for crimping the crimp tube, a stop setting a gap between the jaws when the first and second handles close, and means for adjusting the gap between the jaws when the first and second handles close. In one example, the means for adjusting the gap between the jaws includes a rotatable dial. In one example, the crimp tube has an outer alignment fiducial and internal suture gripping surfaces.

In one example, the means for adjusting includes a wheel or button which changes the length or position of the stop relative to the second handle.

Also featured is a method of deforming a suture crimp tube with an outer alignment fiducial and internal suture gripping surfaces. A suture size is chosen. A fixed wall thickness crimp tube is chosen based on the suture size. A crimper tool with a variable stop and an alignment fiducial is set to the suture size. The chosen suture is placed through the chosen crimp tube. The crimp tube is crimped using the set crimper tool after the alignment fiducial of the crimp tube is aligned with the alignment fiducial of the crimp tool.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a schematic view showing an example of a new medical/veterinary suture crimp tool;

FIG. 2 is a schematic cross-sectional view of the crimp tool of FIG. 1;

FIG. 3 is a schematic top view showing the crimp tool jaws each with a plurality of crimp members;

FIG. 4 is a schematic view showing an example of a preferred crimp tube for use with the crimp tool;

FIG. 5 is a schematic view showing a crimp tube, with two suture ends therein placed between the crimp tool jaws and ready for crimping;

FIG. 6 is a schematic view showing the crimping action of the crimp tool.

FIG. 7 is a schematic three dimensional view of a new crimp tube;

FIG. 8 is a schematic three-dimensional view of an embodiment of a new adjustable crimper tool;

FIG. 9 is a schematic view showing the primary components associated with the stop and an adjustment mechanism for the crimp tool of FIG. 8;

FIG. 10A shows an example of a deformed crimp tube about two side by side 100 lb sutures when the crimp tool of FIG. 8 is used;

FIG. 10B is a schematic view showing a deformed crimp tube about two side by side 20 lb sutures when the crimp tool of FIG. 8 is used;

FIG. 11 is a schematic cross sectional side view of the crimp tool of FIG. 8;

FIG. 12 is a schematic view of the under side of the adjustable dial of the crimp tool of FIG. 8;

FIG. 13 is a schematic view showing the crimp tool of FIG. 8 set for a 20 lb suture;

FIG. 14A-14D show the relative distances between the crimp tool jaws for different adjustable stop settings;

FIG. 15 is a schematic view showing a crimper tool with an extended nose section;

FIG. 16 is a schematic view showing another example of an adjustable stop for a crimper tool; and

FIG. 17 is a schematic view showing another example of an adjustable stop for a crimper tool;

FIG. 18 is a schematic partially cut away view showing an internally threaded crimp tube;

FIG. 19 is a schematic view showing the internal diameter of the threaded crimp tube of FIG. 18; and

FIG. 20 is a flow chart depicting the primary steps associated with forming the internally threaded crimp tube of FIGS. 17 and 18.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

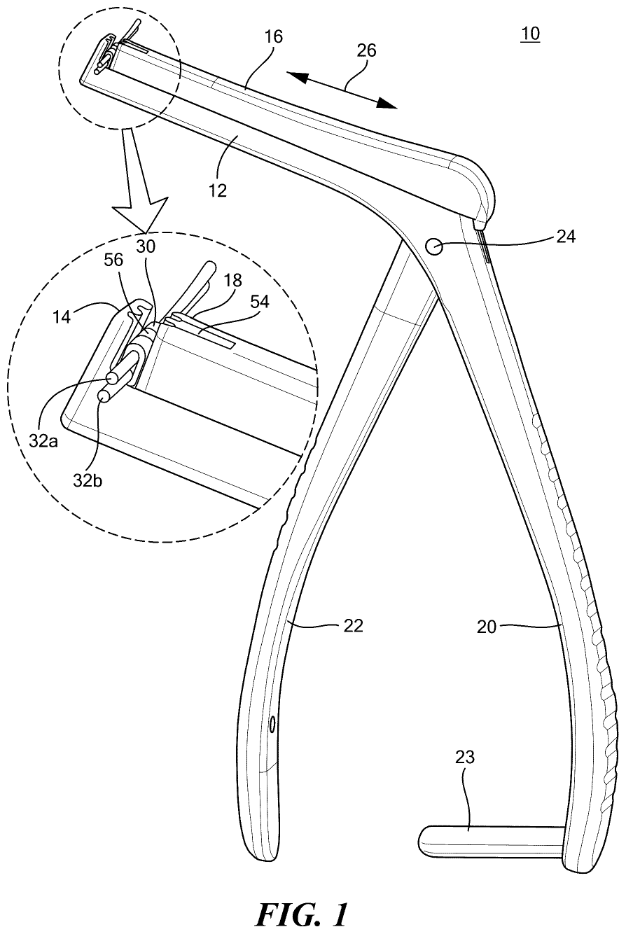

Shown in FIG. 1 is an example of a medical crimper 10 including fixed shank 12 with stationary jaw 14 at the distal end of shank 12 and linearly driven crossbar 16 with moveable jaw 18 at the distal end of crossbar 16. Handle 20 is connected to fixed shank 12 and handle 22 is pivotably connected to fixed shank 12 at pin 24 and is configured to drive crossbar 16 linearly with respect to fixed shank 12 as shown by arrow 26 to fully crimp crimp tube 30 to secure suture ends 32a and 32b passed into crimp tube 30. Stop member 23 of handle 20 strikes handle 22 and prevents the jaws from closing any further so as not to over-crimp a crimp tube. This stop member, however, could be associated with jaw 18, for example.

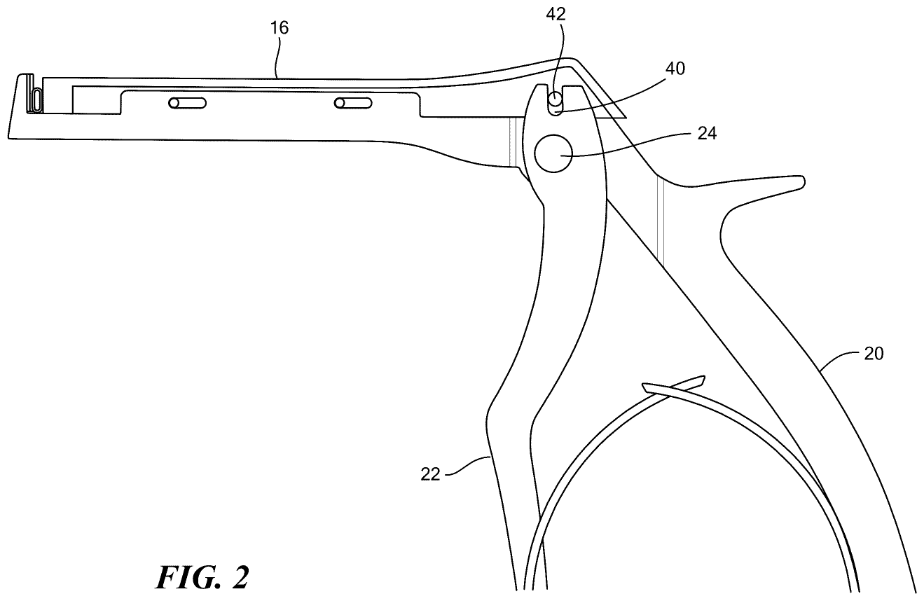

In one example, handle 22, FIG. 2, includes distal socket 40 about drive member 42 extending outward from the side of crossbar 16 to drive the crossbar when handle 22 is closed relative to handle 20. Other means for driving crossbar 16 via handle 22 are possible.

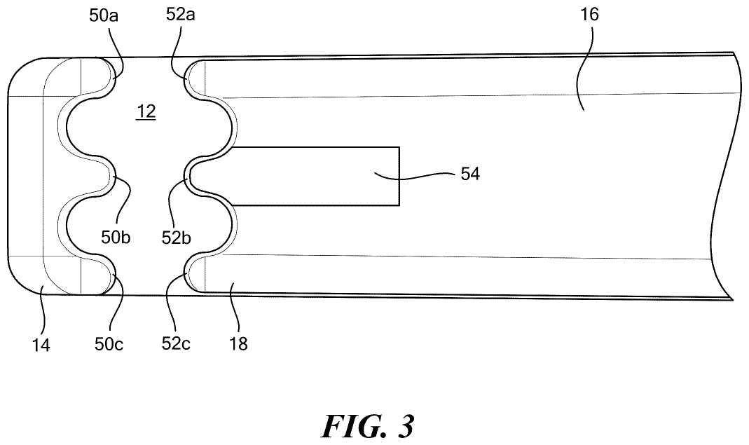

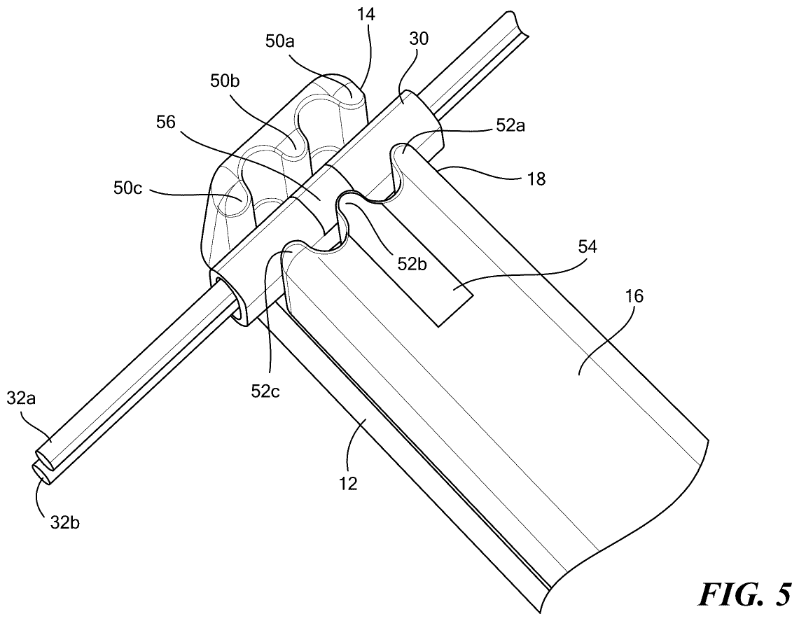

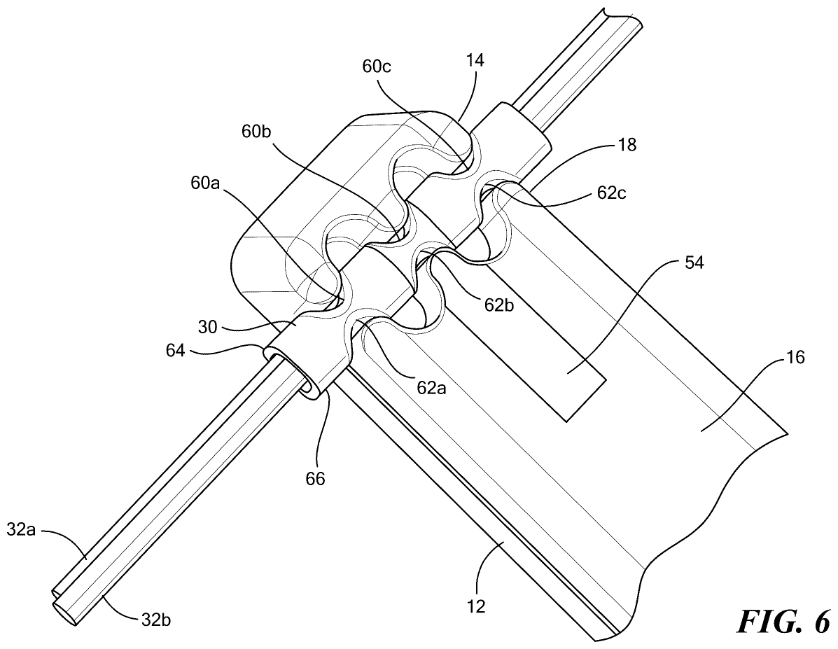

As shown in FIG. 3 (and FIGS. 5-6), stationary jaw 14 includes, across its width and extending at least partially along its length, spaced fixed crimp members 50a, 50b, and 50c and moveable jaw 18 includes, across its width and at least partially across its length spaced crimp members 52a, 52b, and 52c which oppose, respectively, crimp members 50a, 50b, and 50c of jaw 14.

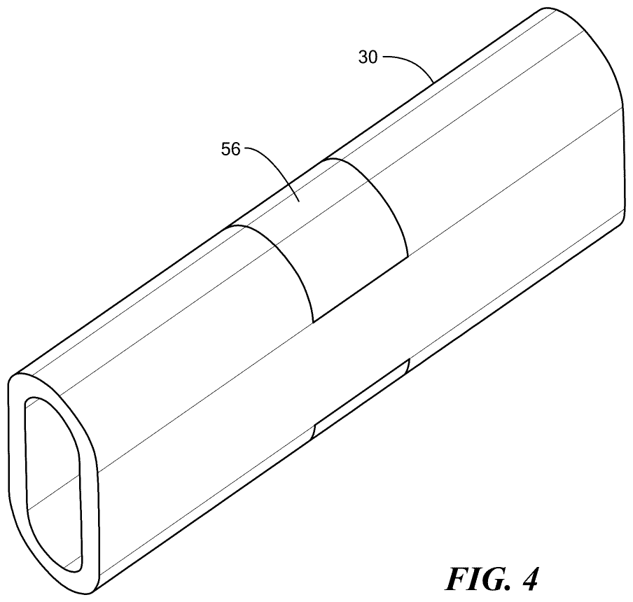

Here, alignment fiducial 54 (e.g., a marker such as a stripe) is associated with crimp member 52b for alignment of a corresponding central alignment fiducial 56 (e.g., 06″ wide) of crimp tube 30, FIG. 4 in order to guide the user as to the proper central position for the crimp tube in the crimper tool when fiducials 54 and 56 align for proper crimping of the crimp tube as shown in FIG. 5. In this example, alignment fiducial 54 extends along crossbar 16 to and then along the face of central crimp member 52b and crimp tube 30 alignment fiducial 56 is centrally located along the extent (length) of the outside of crimp tube 30. The fiducials can be formed using laser marking.

As shown in FIG. 4, crimp tube 30 is preferably an 11-gauge regular wall tube preformed into an oval shape and has a preferred wall thickness of approximately 0.020 inches and formed from a biocompatible, ductile material, e.g., 316L stainless steel. The crimp tube is dimensioned so as to allow two legs of suture through without allowing them to cross inside. A 100 lb crimp was 0.35″ long, between 0.087-0.093″ tall, 0.150″ wide, and with a wall thickness of between 0.0115-0.0145″. A 20 lb crimp was 0.35″ long, between 0.055-0.061″ tall, and 0.180″ wide. Other dimensions are possible. In one preferred embodiment, the internal bore of crimp tube 30 is threaded as shown at 31 for better gripping suture(s). The resulting internal diameter ribbing forming suture gripping surfaces can be formed using a tap (e.g., UNC 3-48) to a depth of 0.003″ minimum.

This preferred shape for the crimp tube and these preferred dimensions make for easier crimping, easier viewability, and the like as shown in FIG. 6 where the crimp pattern in crimp tube 30 are three spaced crimps with each crimp having opposing deformations across the width of the crimp tube. Three deformations 60a, 60b, and 60c are in the mostly flat top side 64 and three deformations 62a, 62b, and 62c are in the bottom flat side 66 of the crimp tube.

The preferred Ronguer style handle crimp tool allows better visibility than plier type handles and still applies enough force to provide a strong crimping action for the sutures placed in the crimp tube. In the preferred embodiment, the wall thickness of the crimp tube is reduced and the crimp tube is partially pre-crimped into the oval shape shown allowing the modified Ronguer handle to be employed to impart multiple crimps at once in the crimp tube with better visibility and still the holding strength of the crimp tube is adequate and in some cases even better because the pre-crimped tube avoids crossing of the sutures within the tube.

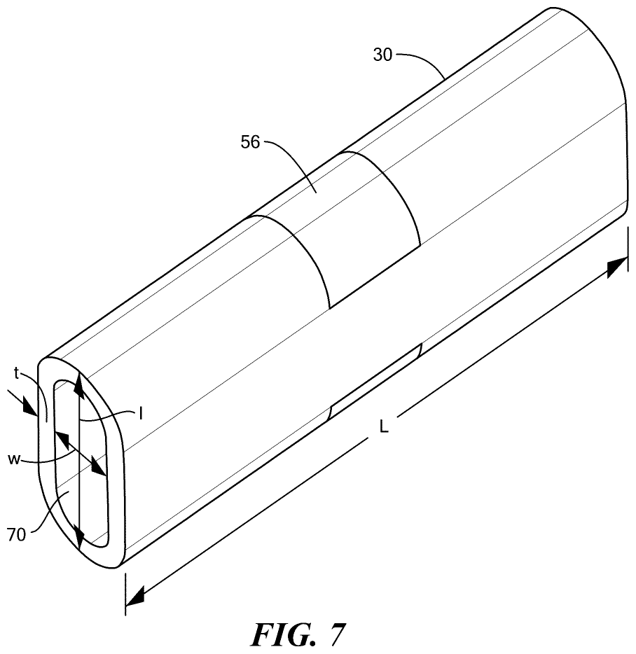

In one embodiment, new crimp tube 30, FIG. 7, has a fixed wall thickness (t) and a variable size opening 70 based on the chosen suture size. Example are listed in table 1 below:

TABLE 1

| | Crimp | Crimp tube | Pinch Point |
|---|---|---|---|
| | tube wall | opening | post |
| Suture size | thickness | height | crimping |
| 20 lb (.022″ dia.) | .0145″ | h = .047″ | 042″ |
| 40 lb (.032″ dia.) | .0145″ | h = .057″ | .048″ |
| 80 lb (.045″ dia.) | .0145″ | h = .069″ | .057″ |
| 100 lb (.055″ dia.) | .0145″ | h = .079″ | .064″ |

In general, the opening allows two sutures to freely pass therethrough but prevents them from crossing. The crimp tubes may be made from TIRW gauge tubing.

The length (l) of each crimp tube may be 0.350″, and the crimp tube may be made of 316 L SS (stainless steel). 304SS or Titanium could be used.

The fairly thin crimp tube wall thickness enables easier crimping for a variety suture sizes.

In one example, once the suture size is chosen, the appropriate crimp tube is chosen corresponding to the chosen suture size. So, for a 100 lb suture, a 100 lb crimp tube is chosen per Table 1 above.

Then, a crimper tool with a variable stop is used to crimp the crimp tube to secure the suture(s) therein.

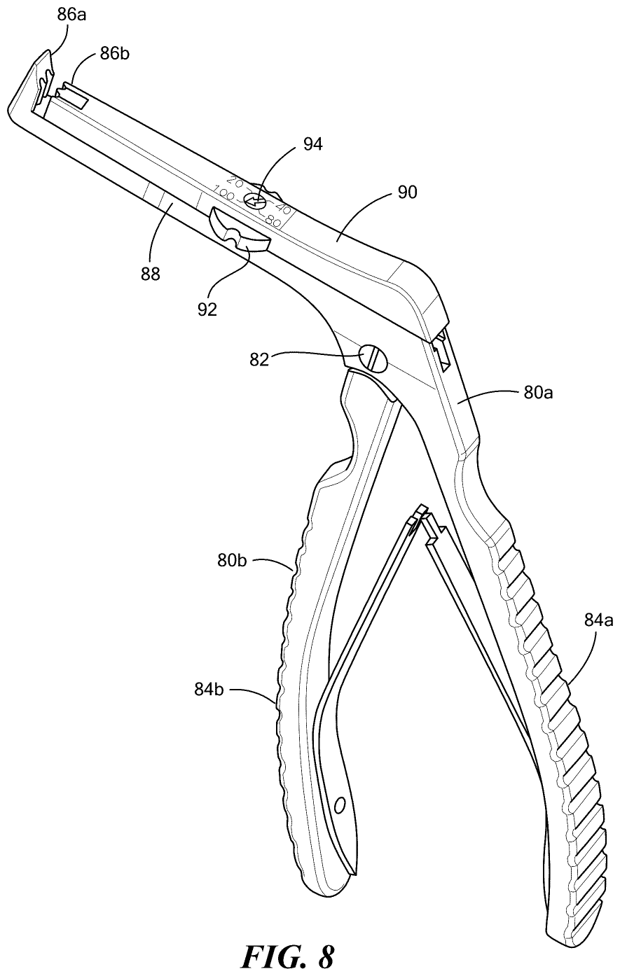

FIG. 8 shows an example of a crimper tool with a variable stop. First arm 80a is coupled the second arm 80b at fastener 82. First arm 80a includes proximal first handle 84a and distal first jaw 86a. Second arm 80b includes proximal second handle 84b and distal second jaw 86b for crimping a crimp tube between jaws 86a and 86b. In this example, the Ronguer style crimper has the stationary jaw 86a located at the end of fixed shank 88 of arm 80a and movable jaw 86b located at the end of linearly driven crossbar 90 of arm 80b. Each jaw preferably has multiple spaced crimp members as discussed earlier. Other style crimpers and other style jaws are possible.

A mechanism such as dial 92 is used to adjust the extent of travel of arm 80a, 80b relative to each other (e.g. by adjusting the extent of travel of crossbar 90) to vary the level of crimp tube deformation. Dial 92 includes settings for 20, 40, 80, and 1001b sutures as shown at 94. In general, the larger (and stronger) the chosen suture, the more distance between the jaws 86a, 86b when the handles are closed and the stop is encountered. The smaller the suture, the distance between the jaws is decreased when the handles are fully closed and the stop is encountered.

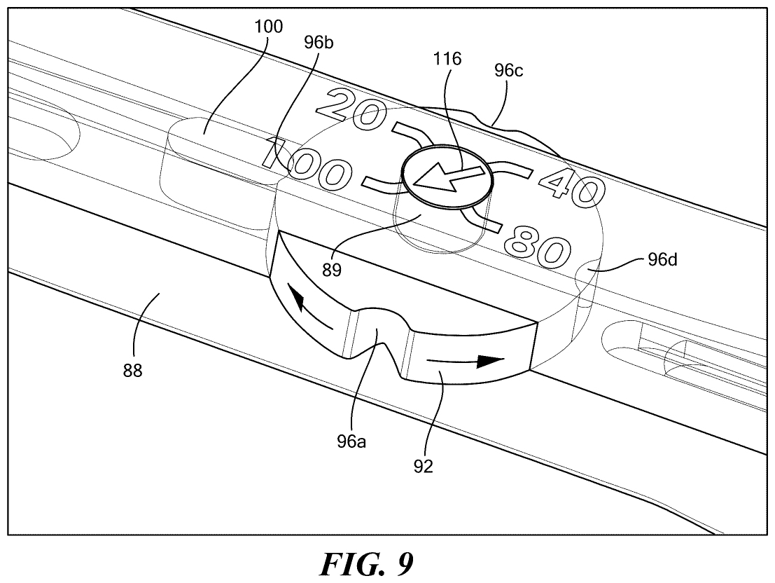

FIG. 9 shows stop 100 on top of fixed shank 88 and dial 92 which travels with the linearly driven crossbar 90 (FIG. 8). Central post 89 of dial 92 extends upward through an opening in crossbar 90 so dial 92 rotates relative to the cross to adjust the crimper jaw spacing when handles are fully closed. Dial 92 cooperates with stop 100 and includes different depth edge recesses 96a-d which each accept stop 100 therein to a different extent thus varying the amount of travel of the linearly driven crossbar which, in turn, adjusts the distance between jaw 86a, 86b, FIG. 8 once handles 84a, 84b are fully closed. The level of crimp tube deformation is usually constant.

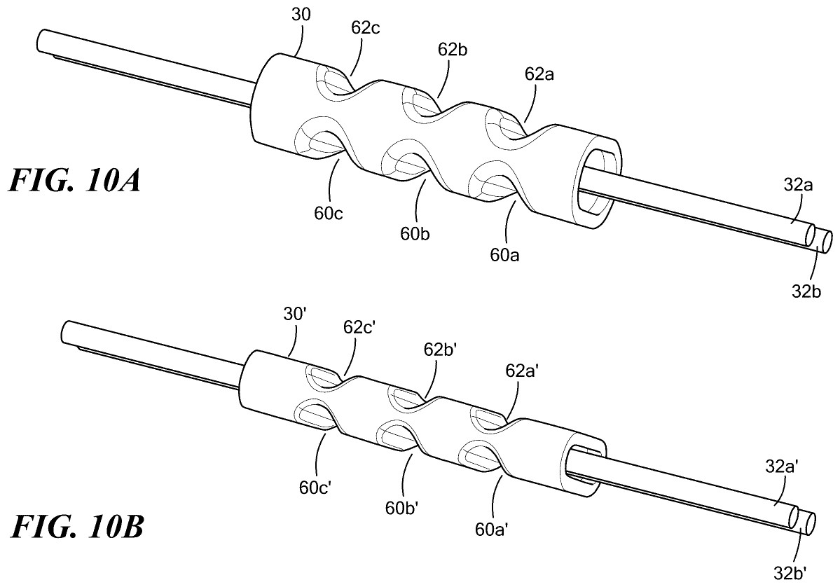

In FIG. 9, dial 92 is set for a 100 lb suture as shown by arrow 116 when dial 92 is rotated about post 89 and thus as recess 96b (the shallowest recess) engages stop 100 when the crimper handles are closed. See FIG. 10A with two side by side 100 lb sutures 32a, 32b crimped in crimp tube 30 by crimp tube defamation 60a-c and 62a-c. For smaller suture sizes, e.g., 80 lb, 40 lb, 20 lb, the crimp tube deformations caused by the deeper edge recesses in the dial 92, FIG. 9 engaging stop 100 decreasing the distance between the crimper jaw when the crimper handles are fully closed. FIG. 10B, for example shows 20 lb sutures 32a′, 32b′ crimped in crimp tube 30′ by crimp deformations 60a′-60c′, and 62a′-62c′.

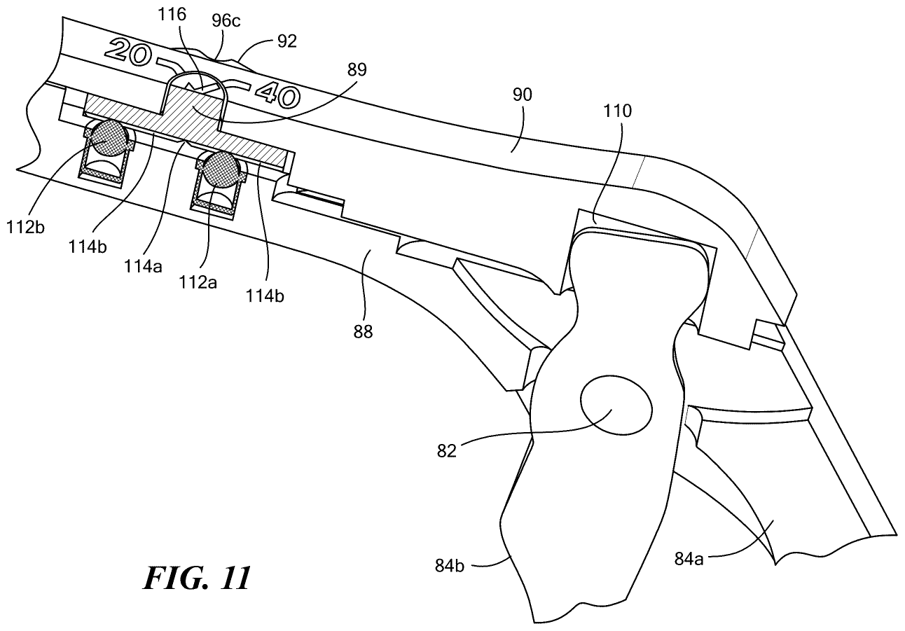

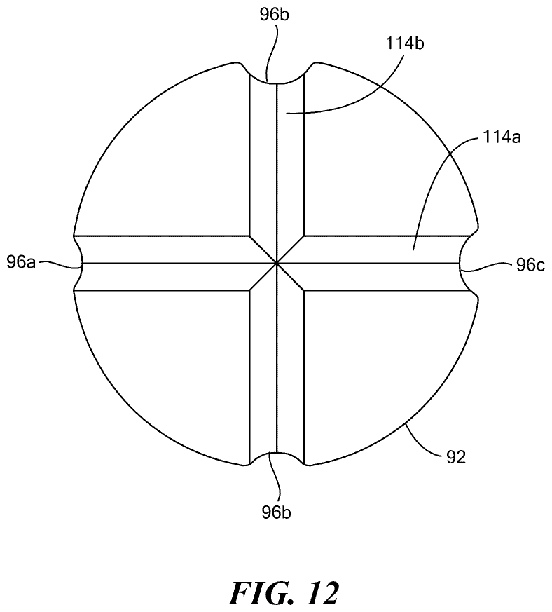

FIG. 11 shows an example of handle 84b pivotably attached to handle 84a at 82 to linearly drive cross bar 90 via the distal end of handle 84b fitted in socket 110 in crossbar 90. Also, spring biased bearings 112a, 112b set in the top of fixed shank 88 cooperated with indentations 114a, 114b in the bottom of dial 92 providing tactile feedback that the dial is set as indicated by pointer 116. See also FIG. 12.

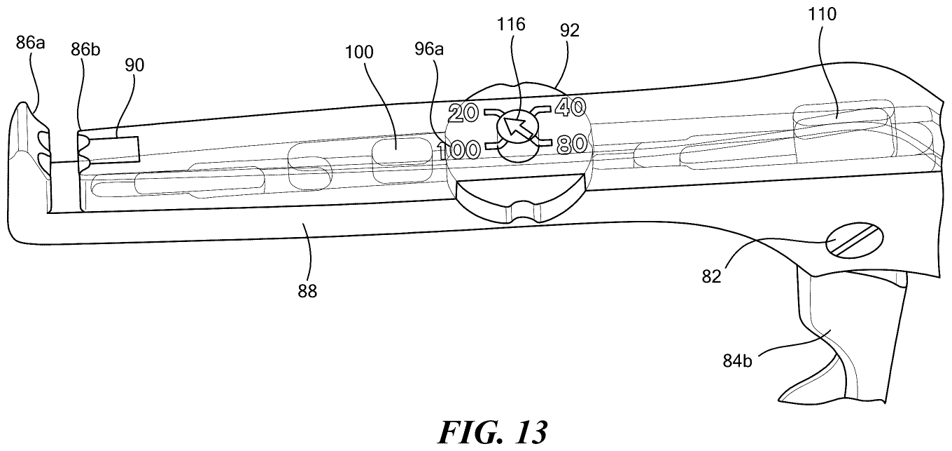

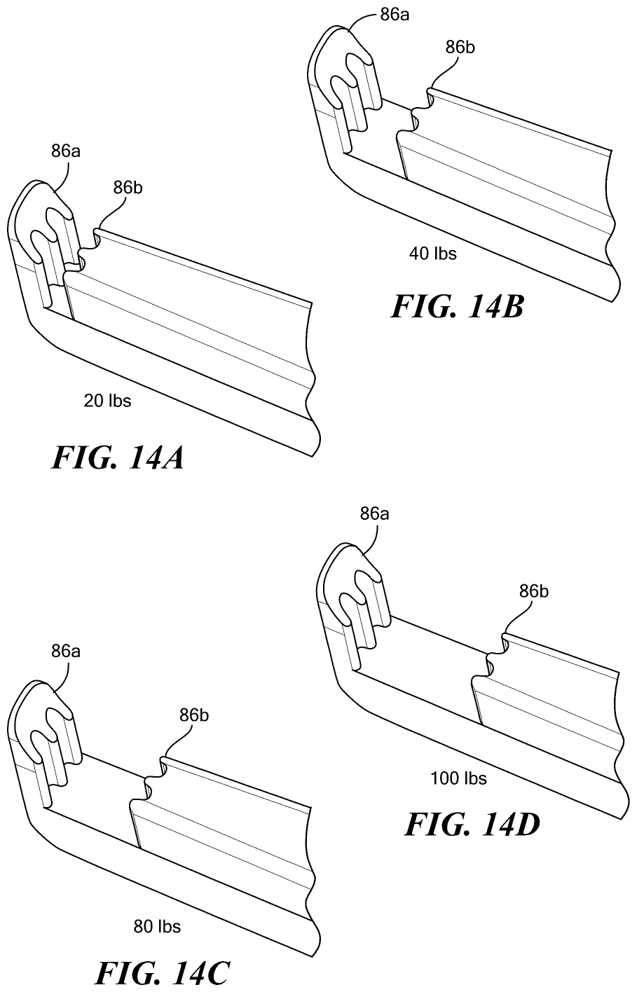

FIG. 13 shows how, when the user sets dial 92 at 20 for a 20 lb suture and a 20 lb crimp tube, crossbar 90 travels further when the handles are squeezed until the deepest dial edge recesses 96a engages stop 100 allowing jaws 86a, 86b to be closer together at the full closing of the crimper handles resulting in deeper crimp tube deformations. See also FIG. 10B. See also FIGS. 14A-14D showing the relative (but not to scale) distances between the crimper tool jaws when the handles are closed and the stop is encountered for different stop settings and different crimp tubes.

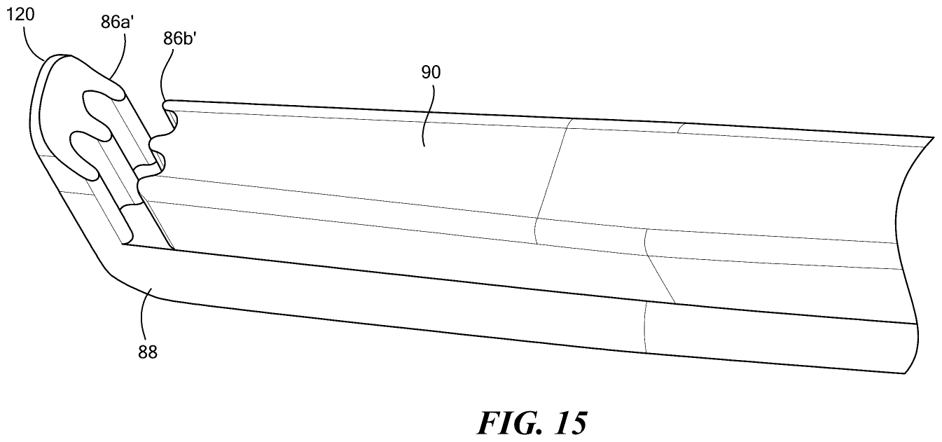

FIG. 15 shows a crimper jaw 86′ with an upwardly extending nose portion 120 functioning to assist in the positioning jaw 86a′ and 86b′ under a tensioned suture during surgery to then crimp the suture. Other configurations of nose extension 120 are possible.

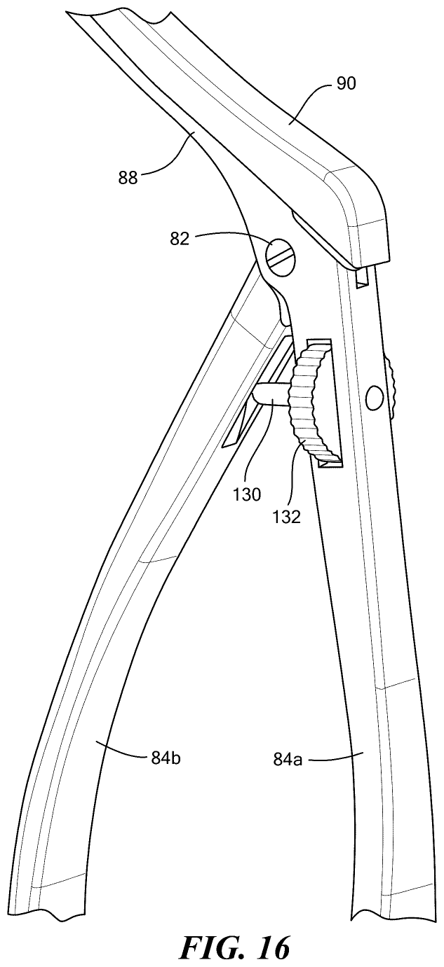

FIG. 16 shows an example of a crimper with stop 130 extending inwardly from handle 84a towards handle 84b and dial 132 which, as it is turns in one direction, increases the length of stop 130 and which, when turned in the opposite direction, decreases the length of stop 130. In this way, the extent to which the handles can close is governed by stop 130 which also adjusts the gap between the crimper jaws when the handles are fully closed.

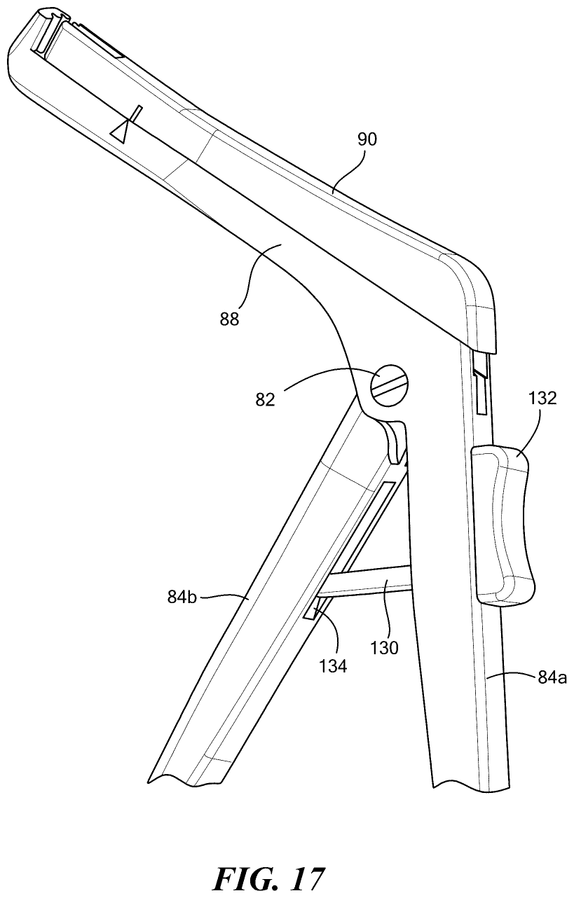

FIG. 17 shows another example with stop 130 extending inwardly from handle 84a toward handle 84b and button mechanism 132 which changes the position of the distal end 134 of stop 130 relative to handle 84b to also adjust the extent to which the handles can close and consequently the distance between the crimper tool jaws when the crimper handles are fully closed.

As a result, especially when different size crimp tubes all have the same thin wall thickness to enable easier crimping to better ensure a proper crimp about the suture(s), the crimp tool has a variable stop set by the practitioner to match the chosen suture (and crimp tube) size.

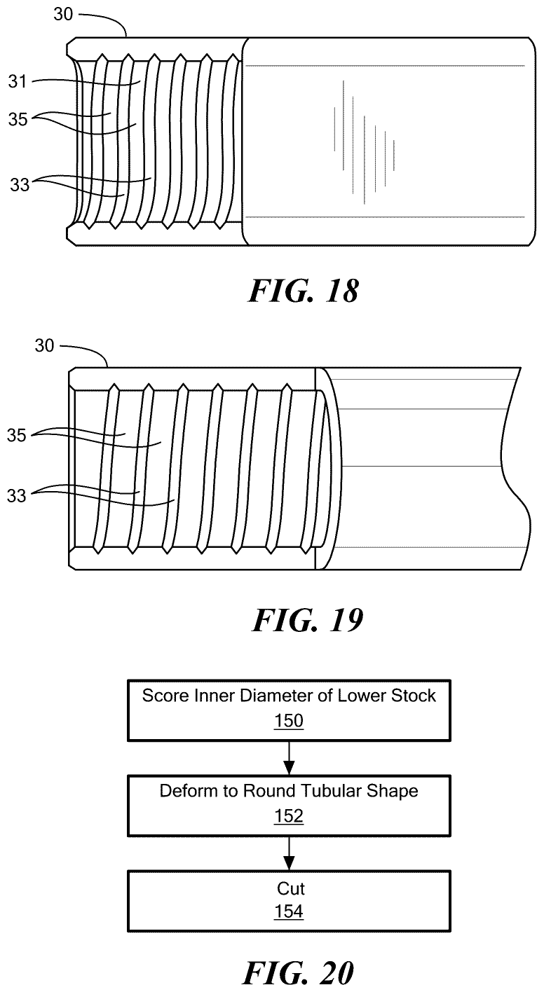

FIGS. 18-19 also show internally threaded crimp tube 30 where a tap or other tool is used to cut or score the inside diameter of the crimp tube resulting in score lines 33 alternating with gripping surfaces 35 for better securing a suture or multiple sutures inside a given crimp tube 30.

In one example, round crimp tube stock is subject to an internal diameter scoring process, step 150, FIG. 20, the stock is preformed into a round tubular shape, step 152, and then cut, step 154, to length to produce multiple oval shaped crimp tubes with internal gripping surfaces.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. An adjustable medical/veterinary suture crimp tool system comprising:
a crimp tool comprising:
a stationary jaw at a distal end of fixed shank,
a moveable jaw at a distal end of a linearly driven crossbar for deforming a crimp tube located between the stationary jaw and the moveable jaw,
an alignment fiducial associated with at least one said jaw,
a first handle connected to a fixed shank,
a second handle pivotably connected to the fixed shank and configured to drive the crossbar linearly with respect to the fixed shank to crimp the crimp tube,
a stop configured to limit the extent of travel of the crossbar, and
a mechanism, cooperating with the stop, for adjusting the extent of travel of the crossbar for crimp tube deformation; and
said crimp tube including internal gripping surfaces and an outer alignment fiducial for alignment with the alignment fiducial of the at least one said jaw of the crimp tool.

2. The crimp tool system of claim 1 in which the stop is on the fixed shank.

3. The crimp tool system of claim 2 in which the mechanism includes a dial on the crossbar with edge recesses of different depths which engage the stop.

4. The crimp tool system of claim 3 in which the dial includes a central post extending through the linearly driven crossbar.

5. The crimp tool system of claim 3 in which the fixed shank includes at least one spring loaded bearing and the dial includes one or more indentations receiving the spring loaded bearing therein for providing tactile feedback.

6. The crimp tool system of claim 1 in which at least one jaw includes an upwardly extending nose extension.

7. The crimp tool system of claim 1 in which the stop is on the first handle and extends inwardly towards the second handle.

8. The crimp tool system of claim 7 in which the mechanism is a wheel which changes the length of the stop relative to the second handle.

9. The crimp tool system of claim 7 in which the mechanism is a button which changes the position of the stop relative to the second handle.

10. The crimp tool system of claim 1 in which the stationary jaw at the distal end of the fixed shank has across its width and extending at least partially along its length spaced fixed crimp members including at least first, second, and third crimp members and the movable jaw at the distal end of the linearly driven crossbar has across its width and extending at least partially along its length, spaced crimp members including at least fourth, fifth, and sixth crimp members opposing, respectively the first, second, and third crimp members of the stationary jaw.

11. The crimp tool system of claim 10 in which the crimp tool alignment fiducial is associated with said fifth crimp member of the moveable jaw.

12. An adjustable medical/veterinary suture crimp tool system comprising:
a crimp tool including:
a first arm having a proximal first handle and a distal first jaw for crimping a crimp tube,
a second arm pivotably coupled to the first arm and having a proximal second handle and a distal second jaw for crimping the crimp tube,
an alignment fiducial associated with at least one said jaw,
a stop configured to limit the extent of travel of the first arm relative to the second arm, and
a mechanism, cooperating with the stop and configured to adjust the extent of travel of the first arm relative to the second arm to for crimp tube deformation; and
said crimp tube including internal suture gripping members and an outer alignment fiducial for alignment with the alignment fiducial of said at least one jaw of the crimp tool.

13. The crimp tool system of claim 12 in which the first arm includes a fixed shank terminating in the first jaw and the second arm includes a linearly driven crossbar terminating in the second jaw.

14. The crimp tool system of claim 13 in which the stop is on the fixed shank.

15. The crimp tool system of claim 14 in which the mechanism includes a dial on the crossbar with edge recesses of different depths which engage the stop.

16. The crimp tool system of claim 15 in which the dial includes a central post extending through the linearly driven crossbar.

17. The crimp tool system of claim 15 in which the fixed shank includes at least one spring loaded bearing and the dial includes at least one indentation receiving the spring loaded bearing therein for providing tactile feedback.

18. The crimp tool system of claim 12 in which the at least one jaw includes an upwardly extending nose extension.

19. The crimp tool system of claim 12 in which the stop is on the first handle.

20. The crimp tool system of claim 19 in which the mechanism is a wheel or button which changes the length or position of the stop relative to the second handle.

21. The crimp tool system of claim 12 in which the first jaw has across its width and extending at least partially along its length spaced fixed crimp members including at least first, second, and third crimp members and the second jaw has a cross its width and extending at least partially along its length spaced crimp members including at least fourth, fifth, and sixth crimp members opposing, respectively the first, second, and third crimp members of the first jaw.

22. The crimp tool system of claim 21 in which the crimp tool alignment fiducial is associated with the fifth crimp member of the second jaw.

23. An adjustable/medical veterinary suture crimp tool system comprising:
a crimp tool including:
a first arm having a proximal first handle and a distal first jaw for crimping a crimp tube,
a second arm pivotably coupled to the first arm and having a proximal second handle and a distal second jaw for crimping the crimp tube,
an alignment fiducial associated with at least one said jaw,
a stop setting a gap between the jaws when the first and second handles close, and
means for adjusting the gap between the jaws when the first and second handles close; and
the crimp tube including internal suture gripping members and an outer alignment fiducial for alignment with the fiducial of said at least one jaw of the crimp tool.

24. The crimp tool system of claim 23 in which the means for adjusting the gap between the jaws includes a rotatable dial.

25. The crimp tool system of claim 23 in which the stop is located on a fixed shank of the first arm.

26. The crimp tool of claim 25 in which the means for adjusting includes a dial on a crossbar of the second arm.

27. The crimp tool system of claim 23 in which the at least one jaw includes an upwardly extending nose extension.

28. The crimp tool system of claim 23 in which the stop is on the first handle and extends inwardly towards the second handle.

29. The crimp tool system of claim 28 in which the means for adjusting includes a wheel or button which changes the length or position of the stop relative to the second handle.

30. The crimp tool system of claim 23 in which the first jaw includes at least first, second, and third crimp members and the second jaw includes at least fourth, fifth, and sixth crimp members opposing, respectively, the first, second, and third crimp members of the first jaw.

31. The crimp tool system of claim 23 in which the alignment fiducial of the crimp tool is associated with the fifth crimp member of the second jaw.

32. A method of deforming a suture crimp tube, the method comprising:
choosing a suture size;
choosing a fixed wall thickness internally threaded crimp tube based on the suture size;
setting a crimper tool with a variable stop to the suture size;
placing the chosen suture through the chosen crimp tube;
aligning an alignment fiducial of the crimper tool with an alignment fiducial of the crimp tube; and
crimping the crimp tube using the set crimper tool.
